(19) United States
(12) Patent Application Publication
(10) Pub. No.: US 2024/0042259 A1
(43) Pub. Date: Feb. 8, 2024

(54) NERVE AGENT DECONTAMINATING AGENTS

(71) Applicants: V. Prakash Reddy, Rolla, MO (US); Puspa Aryal, Rolla, MO (US)

(72) Inventors: V. Prakash Reddy, Rolla, MO (US); Puspa Aryal, Rolla, MO (US)

(73) Assignee: The Curators of the University of Missouri, Columbia, MO (US)

(21) Appl. No.: 18/363,429

(22) Filed: Aug. 1, 2023

Related U.S. Application Data
(60) Provisional application No. 63/394,055, filed on Aug. 1, 2022.

(51) Int. Cl. A62D 3/35; A61K 31/155; A61K 31/426; A61K 31/44; A61K 31/417

(52) U.S. Cl. CPC A62D 3/35; A61K 31/155; A61K 31/426; A61K 31/44; A61K 31/417; A62D 2101/02

(57) ABSTRACT

Agents for decontaminating and/or neutralizing organophosphorus compounds (e.g., nerve agents, pesticides) and methods of using those agents are disclosed. Preferred agents comprise aminoguanidine imines, and these agents do not require a strongly alkaline environment to be effective. The aminoguanidine imines can hydrolytically decontaminate substantially all of the target organophosphorus compound(s) in a matter of minutes.

### CROSS-REFERENCE TO RELATED APPLICATIONS

The present application claims the priority benefit of U.S. Provisional Patent Application Ser. No. 63/394,055, filed Aug. 1, 2022, entitled NERVE AGENT DECONTAMINATING AGENTS, the entirety of which is incorporated by reference herein.

### FEDERALLY SPONSORED RESEARCH OR DEVELOPMENT

This invention was made with government support under Contract No. W911NF2120259 awarded by the Army Research Laboratory (ARL). The government has certain rights in the invention.

### BACKGROUND

### Field

The present disclosure relates to novel decontaminating agents for organophosphate-based nerve agents and pesticides.

### Description of Related Art

Oxime-based compounds are typically used in nerve agent decontaminating agents and prophylactics against exposure to nerve agents or structurally related pesticides. These decontaminating agents, such as Dekon-139 (i.e., diacetyl monoxime), are used under strongly alkaline conditions and are associated with severe toxicity effects when used for decontaminating the exposed skin surfaces.

There is a need for decontaminating agents that are safer alternatives to the currently existing options, while also being effective at neutralizing the target nerve agent or pesticide.

### SUMMARY

The present disclosure is broadly concerned with decontamination or neutralization methods and products.

In one embodiment, a decontamination or neutralization method comprising contacting an organophosphorous compound with an aminoguanidine imine is provided.

In another embodiment, the disclosure provides a decontamination or neutralization method comprising contacting an aminoguanidine imine with a surface suspected of contamination or that might become contaminated.

In a further embodiment, a decontaminant product comprising an aminoguanidine imine is disclosed.

### BRIEF DESCRIPTION OF THE DRAWINGS

FIG. 1 is a ³¹P NMR spectrum obtained as described in Example 2 (Part 1) that shows the complete disappearance of the methyl paraoxon δ³¹P at −4.89 from a reaction mixture comprising methyl paraoxon and benzaldehyde aminoguanidine imine, and the appearance of the δ³¹P at −0.84, which corresponds to the hydrolyzed product (7);

FIG. 2 is a ³¹P NMR spectrum obtained as described in Example 2 (Part 1) that shows the complete disappearance of the methyl paraoxon δ³¹P at −4.89 from a reaction mixture comprising methyl paraoxon and pyridine-4-adehyde aminoguanidine imine, and the appearance of the δ³¹P at −0.84, which corresponds to the hydrolyzed product (7);

FIG. 3 is a ³¹P NMR spectrum obtained as described in Example 2 (Part 1) that shows the complete disappearance of the methyl paraoxon δ³¹P at −4.89 from a reaction mixture comprising methyl paraoxon and Dekon-139, and the appearance of the δ³¹P at −0.84, which corresponds to the hydrolyzed product (7);

FIG. 4 is a ³¹P NMR spectrum of methyl paraoxon in the absence of decontaminating agents (δ³¹P=−4.89), thus providing support for the conclusions noted in FIGS. 1-3;

FIG. 5 is a ³¹P NMR spectrum obtained as described in Example 2 (Part 2) that shows the complete disappearance of the triphenyl phosphate δ³¹P at −16.28 from a reaction mixture comprising triphenyl phosphate and benzaldehyde aminoguanidine imine, and the appearance of the δ³¹P at −11.84, which corresponds to the hydrolyzed product (6);

FIG. 6 is a ³¹P NMR spectrum obtained as described in Example 2 (Part 2) that shows the complete disappearance of the triphenyl phosphate δ³¹P at −16.28 from a reaction mixture comprising triphenyl phosphate and pyridine-4-aldehyde aminoguanidine imine, and the appearance of the δ³¹P at −12.50 (slightly deshielded absorption, which may be due to the concentration effect), which corresponds to the hydrolyzed product (6);

FIG. 7 is a ³¹P NMR spectrum obtained as described in Example 2 (Part 2) that shows the complete disappearance of the triphenyl phosphate δ³¹P at −16.28 from a reaction mixture comprising triphenyl phosphate and Dekon-139, and the appearance of the δ³¹P at −11.78, which corresponds to the hydrolyzed product (6);

FIG. 8 is a ³¹P NMR spectrum of triphenyl phosphate in the absence of decontaminating agents (δ³¹P=−16.28), thus providing support for the conclusions noted in FIGS. 5-7; and

FIG. 9 is a ³¹P NMR spectrum obtained as described in Example 2 (Part 3) that shows the disappearance over time of the triphenyl phosphate δ³¹P at −16.28 from a reaction mixture comprising triphenyl phosphate and aldehyde imine, along with the appearance over that same time of the δ³¹P at −11.78, which corresponds to the hydrolyzed product (6).

### DETAILED DESCRIPTION

The present disclosure is broadly concerned with decontamination or neutralization methods and products.

In one embodiment, the decontamination or neutralization methods comprise contacting an organophosphorous compound with an aminoguanidine imine. It is preferred that the contact results in a reaction between the organophosphorous compound and the aminoguanidine imine, and more preferably that reaction comprises hydrolyzation of the organophosphorus compound.

Suitable aminoguanidine imines are derived from one or more of aryl (preferably C₆ to C₁₀) aldehydes, heteroaryl (preferably 3 to 10 ring atoms) aldehydes, aliphatic (preferably C₁ to C₁₂, and more preferably C₁ to C₆) aldehydes, or combinations thereof.

Examples of aryl aldehydes from which suitable aminoguanidine imines can be derived include those chosen from benzaldehyde, salicylaldehyde, 3,4-dihydroxybenzaldehyde, 3,4,5-trihydroxybenzaldehyde, or combinations thereof. Suitable heteroaryl aldehydes include those chosen from 4-pyridinecarboxaldehyde, imidazole-5-carboxaldehyde, a thiazole aldehyde, a thiophene aldehyde, or combinations thereof. Aliphatic aldehydes can include those chosen from acetaldehyde, propionaldehyde, or combinations thereof.

In some embodiments, aminoguanidine imines for use herein comprise one or more of an aryl (preferably C₆ to C₁₀) aldehyde aminoguanidine imine, an aliphatic (preferably C₁ to C₁₂, and more preferably C₁ to C₆) aldehyde aminoguanidine imine, a heteroaryl (preferably 3 to 10 ring atoms) aldehyde aminoguanidine imine, or combinations thereof.

Some preferred aminoguanidine imines are chosen from one or more of:

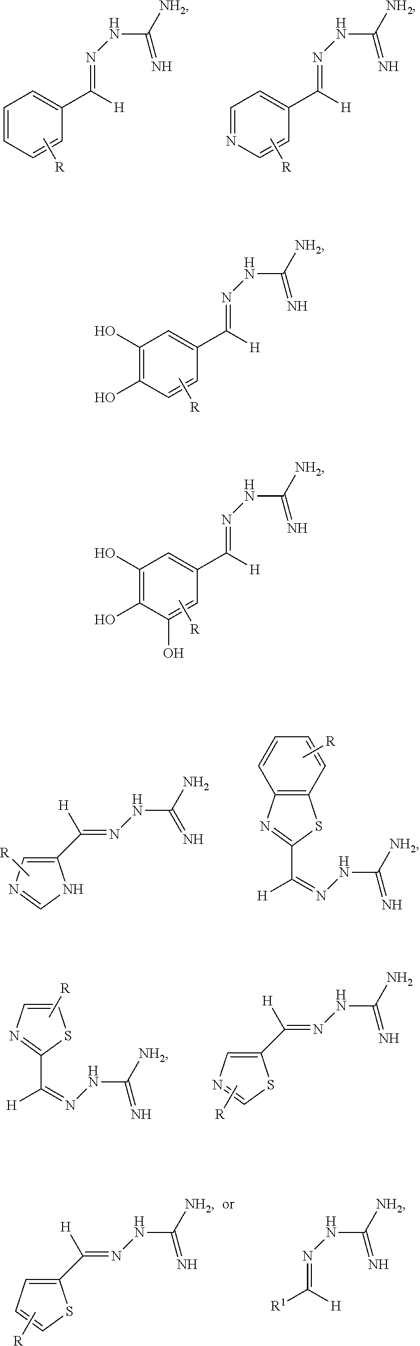

wherein:
R is selected from the group consisting of alkyl (preferably C₁ to C₁₂, and more preferably C₁ to C₆) groups, aryl (preferably C₆ to C₁₀) groups, —H, and —OR²;
R¹ is selected from from the group consisting of alkyl (preferably C₁ to C₁₂, and more preferably C₁ to C₆) groups, aryl (preferably C₆ to C₁₀) groups, —H, —OR², and polymers (e.g., polystyrenes, polynorbornenes, poly(4-vinylpyridine), poly(thiophenes), poly(imidazoles), poly(pyrroles), poly(thiazoles), poly(benzothiazoles); and
R² is selected from the group consisting of —H and alkyl (preferably C₁ to C₁₂, and more preferably C₁ to C₆) groups.

In embodiments where R¹ is a polymer, the aminoguanidine imine moiety is preferably bonded to a monomer of the polymer, either through a functional group on the monomer or directly to the polymer backbone. Examples of aminoguanidine imines where R¹ is a polymer include:

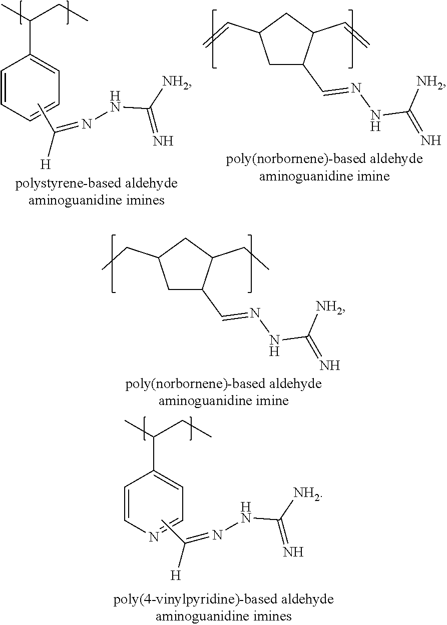

Some preferred aminoguanidine imines include one or more of benzaldehyde aminoguanidine imine, 4-pyridine aldehyde aminoguanidine imine, imidazole-5-carboxaldehyde aminoguanidine imine, 3,4-dihydroxybenzaldehyde aminoguanidine imine, 3,4,5-trihydroxybenzaldehyde aminoguanidine imine, or combinations thereof.

Organophosphorus compounds that can be decontaminated and/or neutralized by the aminoguanidine imines as described herein are preferably organophosphates. Some examples of organophosphorus compound to be decontaminated and/or neutralized include one or more of nerve agents, toxins, pesticides, simulants for pesticides, and/or herbicides. Specific examples include one or more of triphenyl phosphate, methyl paraoxon, paraoxon, chlorpyrifos oxon, malaoxon, sarin (GB), tabun (GA), cyclosarin (GF), VX ((O-ethyl-S-[2(diisopropylamino)ethyl]methylphosphonothioate); nerve agent), and/or GV (2-(dimethylamino)ethyl N,N-dimethylphosphoramidofluoridate; nerve agent).

In some embodiments, the target organophosphorus compound is present on a surface to be decontaminated, and the aminoguanidine imine is contacted with that surface. In one or more embodiments, a surface may be suspected of contamination, either presently or potentially in the future, and that the surface can be treated prophylactically with the aminoguanidine imine(s).

The contacting step can be carried out in a solvent (e.g., dimethyl sulfoxide, dimethyl formamide, and mixtures thereof) in some instances. Additionally or alternatively, decontamination can be effected in the solid phase under hygroscopic conditions.

In some embodiments, about 1 to about 10, and preferably about 1 to about 5 mol equivalents of aminoguanidine imine relative to organophosphorus compound(s) is utilized.

Advantageously, a strongly alkaline environment is not required for the methods disclosed herein to effectively neutralize or decontaminate the organophosphorus compound. That is, contacting of the aminoguanidine imine and organophosphorus compound can be carried out in an environment having a pH of less than about 10.3, preferably less than about 10, more preferably less than about 9.5, and even more preferably less than about 9. In other embodiments, that contact is carried out in an environment having a pH of about 8 to about 10, preferably about 8 to about 9.5, and more preferably about 8.5 to about 9.5.

The contact time can be maintained for any amount of time desired by the user, but advantageously decontamination and/or neutralization can take place with contact that is carried out for less than about 5 minutes, preferably less than about 3 minutes, more preferably less than about 2 minutes, and even more preferably less than about 1 minute.

Regardless of the contact time, at least about 90%, preferably at least about 95%, more preferably at least about 98%, and even more preferably about 100% of organophosphorus compound is reacted during said reacting, as determined by ³¹P NMR spectroscopy. Even more preferably, this % conversion takes place within the time frames set forth in the immediately preceding paragraph, forming a hydrolytic product of the organophosphorus compound.

In some embodiments, the aminoguanidine imine can be provided as part of a solution, lotion, kit, gel, or solid.

Additional advantages of the various embodiments will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the disclosure. It is to be understood, however, that these examples are provided by way of illustration, and nothing therein should be taken as a limitation upon the overall scope.

The following studies were carried out to confirm arylaldehyde-aminoguanidine imines as safer alternatives to current nerve agent decontaminating agents. The aminoguanidine-aldehyde imines, such as those derived from 4-pyridinecarboxaldehyde, quantitatively hydrolyzed the pesticide simulant triphenyl phosphate at pH 9.5 in 1 to 2 minutes. These results showed that aminoguanidine-based imines provide effective nerve agent decontamination, and that they may also be used in prophylactic treatments and/or as therapeutics for nerve-agent or pesticide exposure.

### Example 1

### Experimental Overview

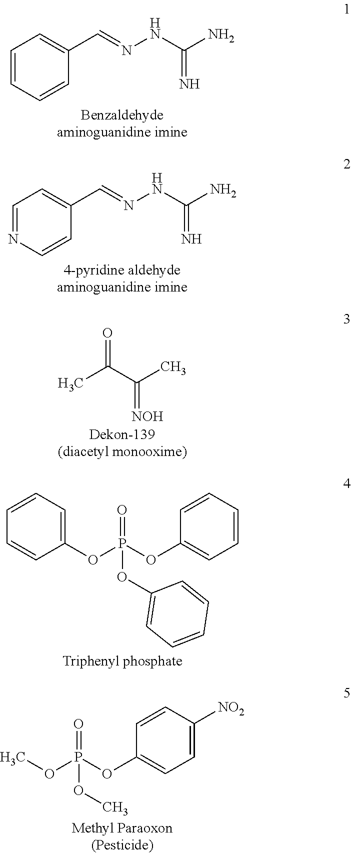

The relative hydrolytic reactivity of triphenyl phosphate (as a model compound for organophosphate pesticides) was compared with that of methyl paraoxon in glycine buffer in dimethyl sulfoxide (“DMSO”; 1 M; 1:1 v/v of buffer to DMSO; pH=9.5), using aryl aldehyde aminoguanidine imines (1 and 2) as the decontaminating agents. DMSO as a cosolvent increases the solubility of the compounds in the reaction mixture. Reaction of the triphenyl phosphate (4) as well as methyl paraoxon (5) with the benzaldehyde aminoguanidine imine (1) or pyridine-4-aldehyde aminoguanidine imine (2) proceeded quantitatively in less than 1 min (˜48 sec) at pH 9.5 (Scheme A). In a separate experiment, it was determined that methyl paraoxon (5) reacted ˜1.2 times faster than the triphenyl phosphate (4).

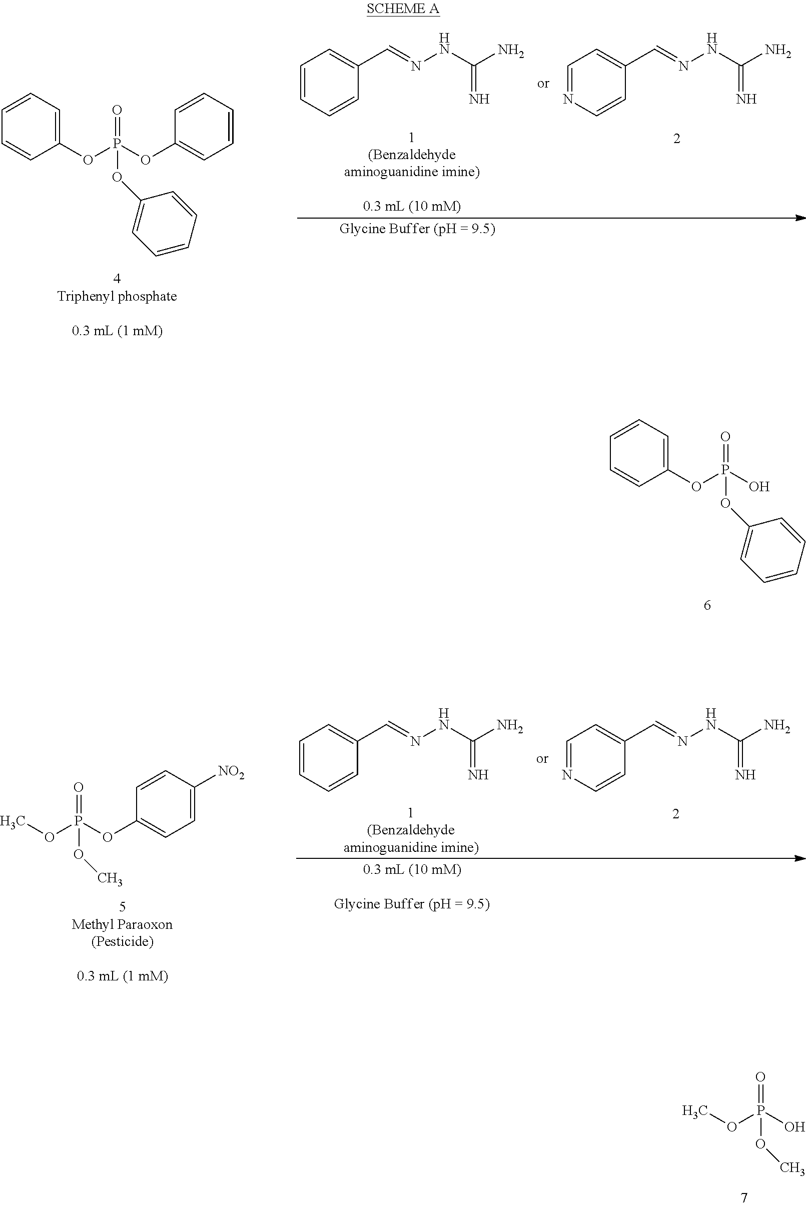

The decontaminating efficiencies of compounds (1), (2), and (3) were followed using ³¹P NMR spectroscopy. The ³¹P NMR chemical shifts of the hydrolytic products (6) and (7) were substantially deshielded as compared to that of the unreacted compounds (4) and (5). The ³¹P NMR spectra were acquired at 161 MHz on a Bruker Avance NMR instrument, using a relaxation delay time of 3 to 4 seconds (>3 T1) for quantitative integration of the peaks. Reaction time was taken as half the total acquisition time of the spectra. As shown in the ³¹P NMR spectra obtained while monitoring the progress of the hydrolytic decontamination reactions, the hydrolysis of the methyl paraoxon as well as triphenyl phosphate was complete in less than 1 min.

Under reaction conditions similar to those above, Dekon-139 (diacetyl monoxime; 3) also gave quantitative conversion of the methyl paraoxon and triphenyl phosphate (used as a pesticide simulant) in less than 1 min (˜48 sec) at pH 9.5 (Scheme B). Thus, the aryl aldehyde aminoguanidine imine compounds (1) and (2) are as effective (or relatively more effective) in the decontamination of the organophosphate pesticides as the current “state-of-the-art” decontaminating agent Dekon-139 (3). The aryl aldehyde aminoguanidine imine derivatives (e.g., 1 and 2) thus provide nontoxic alternatives to Dekon-139.

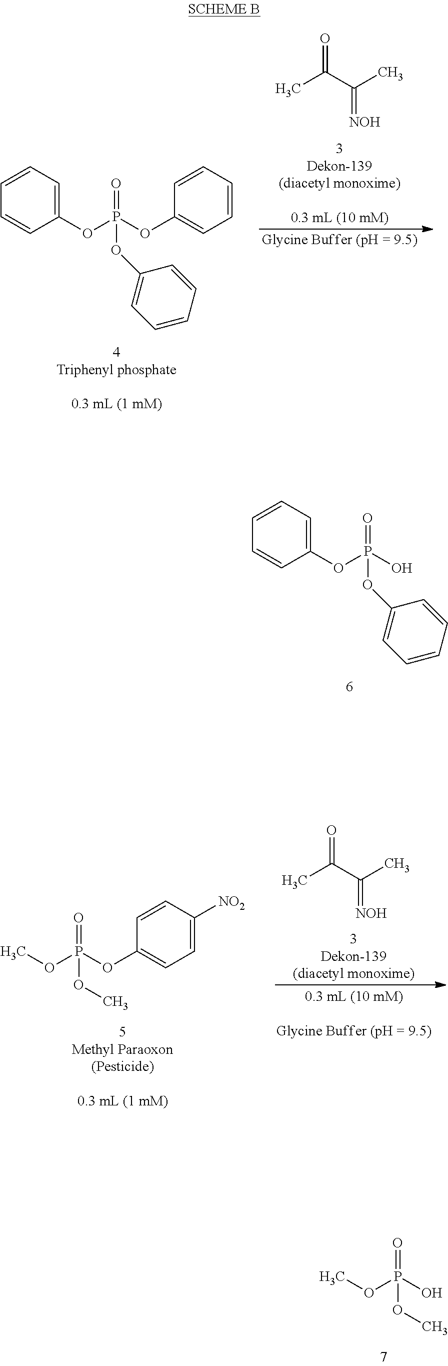

### Example 2

### Experimental and Supporting ³¹P NMR Spectra

1. Hydrolytic Decontamination of Methyl Paraoxon

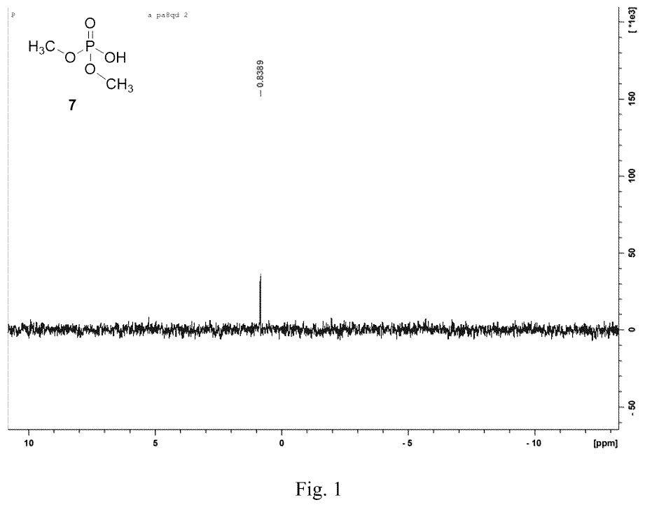

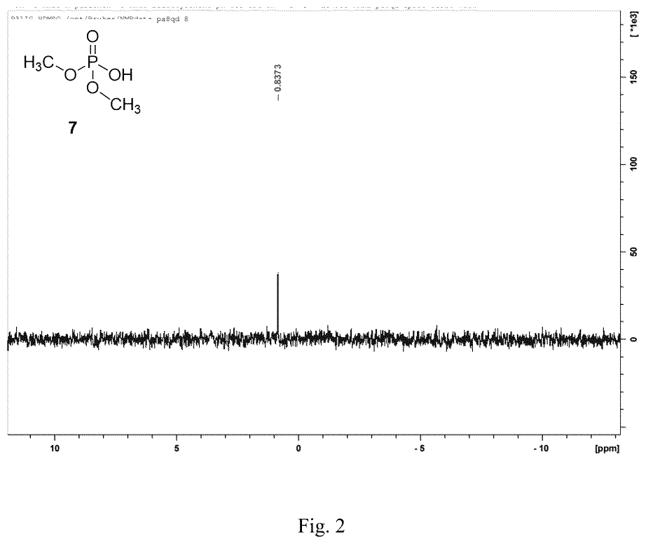

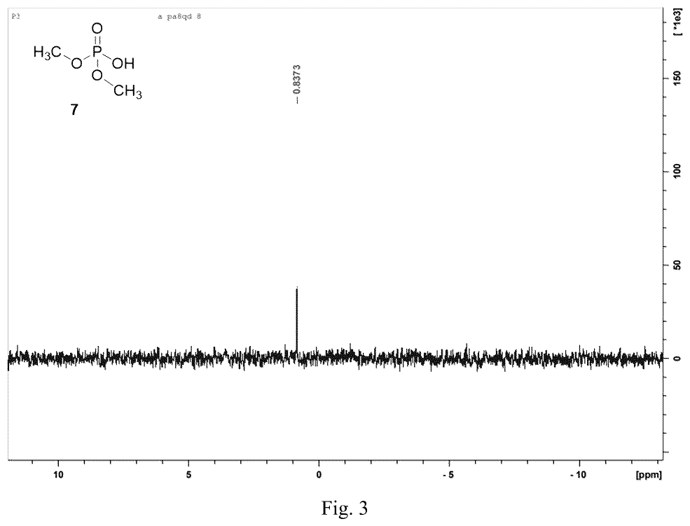

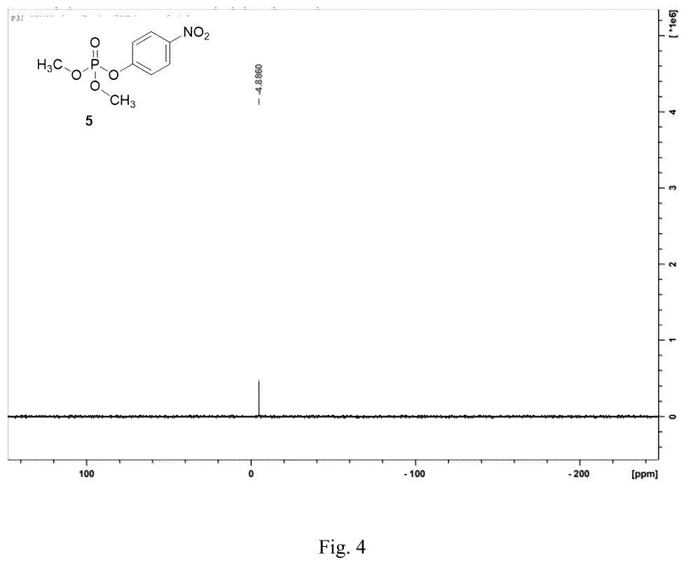

Methyl paraoxon (5; 0.3 mL of 1 mM solution) was mixed with 0.3 mL of 10 mM imine (1; Scheme C, or 2; Scheme D) or Dekon-139 (3; Scheme E) in glycine buffer (H₂O/DMSO) at pH 9.5 in a reaction vial. The mixture was immediately placed in an NMR tube, and the acquisition of the spectra commenced. The spectra were recorded at 25° C. by accumulation of 32 scans. Total acquisition time for the run was 1 min 36 secs. The reaction time was taken to be the midpoint of the acquisition period. The resulting spectra are shown in FIGS. 1-3, and the ³¹P NMR spectrum of methyl paraoxon (5) is shown in FIG. 4.

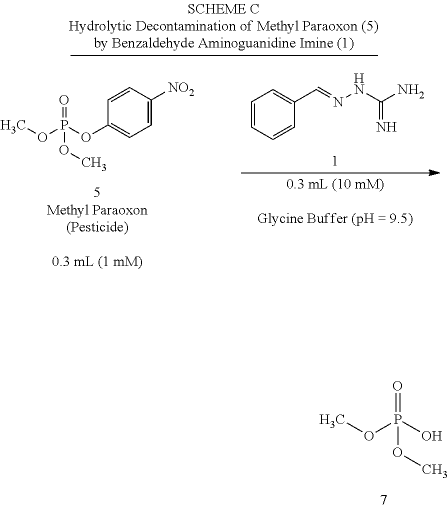

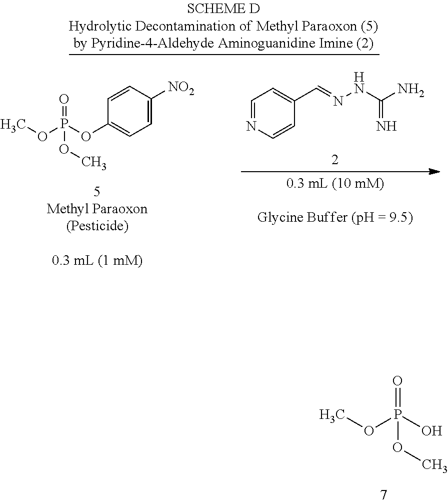

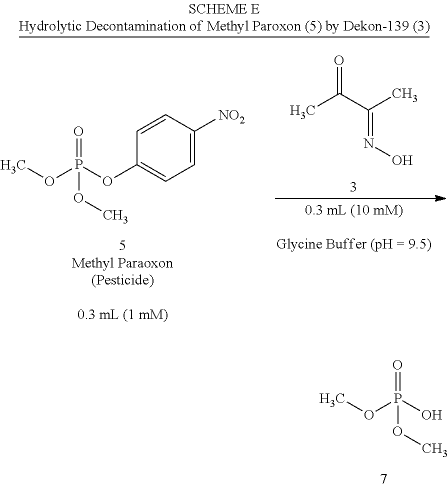

2. Hydrolytic Decontamination of Triphenyl Phosphate

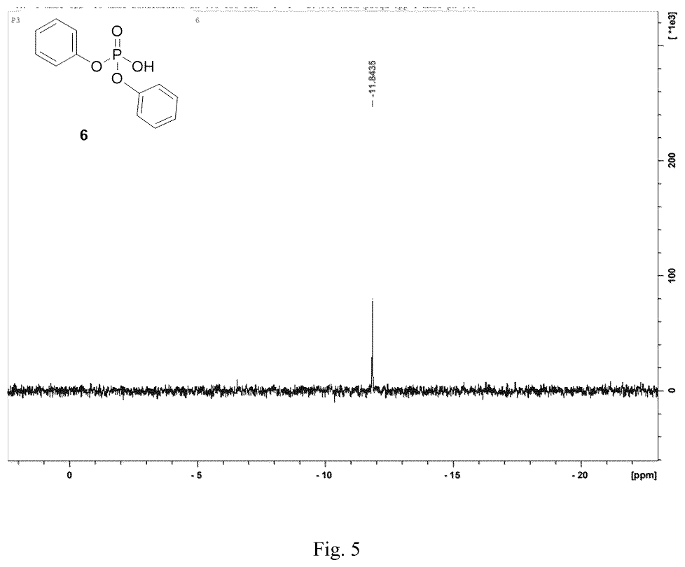

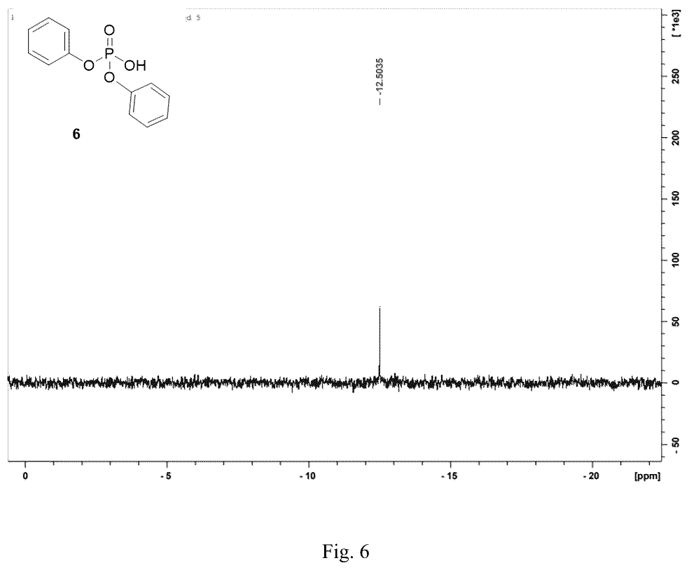

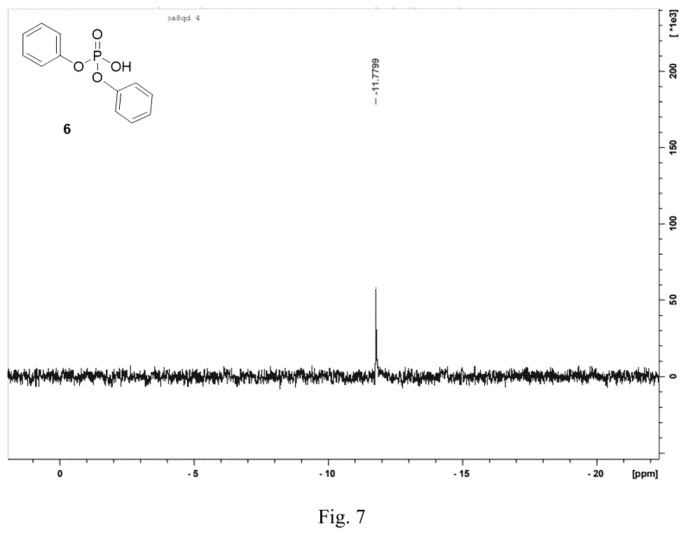

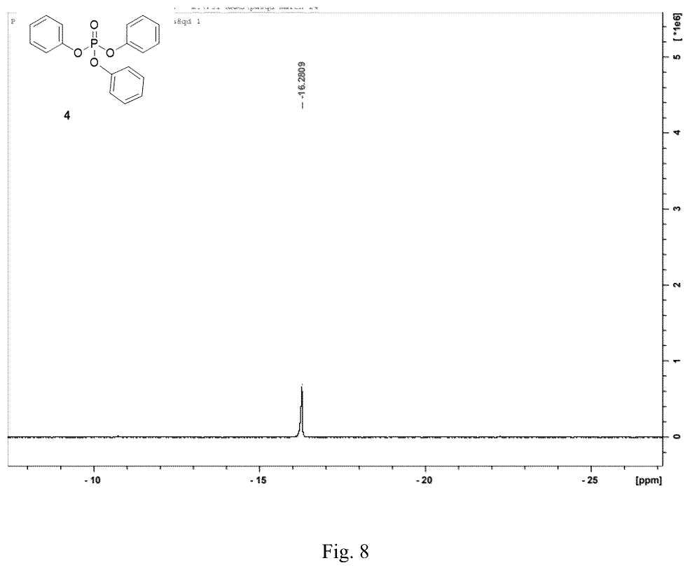

Triphenyl Phosphate (4; 0.3 mL of 1 mM solution) was mixed with 0.3 mL of 10 mM solutions of the aminoguanidine imine (1; Scheme F, or 2; Scheme G), or Dekon-139 (3; Scheme H) in glycine buffer (in H₂O/DMSO) at pH 9.5 in a reaction vial. The mixture was immediately placed in an NMR tube, and the acquisition of the spectra commenced. The spectra were recorded at 25° C. by accumulation of 32 scans. Total acquisition time for the run was 1 min 36 secs. The reaction time was taken to be the midpoint of the acquisition period (approximately 48 secs). The resulting spectra are shown in FIGS. 5-7. FIG. 8 shows the ³¹P NMR spectrum of triphenyl phosphate (4) in glycine buffer pH 9.5.

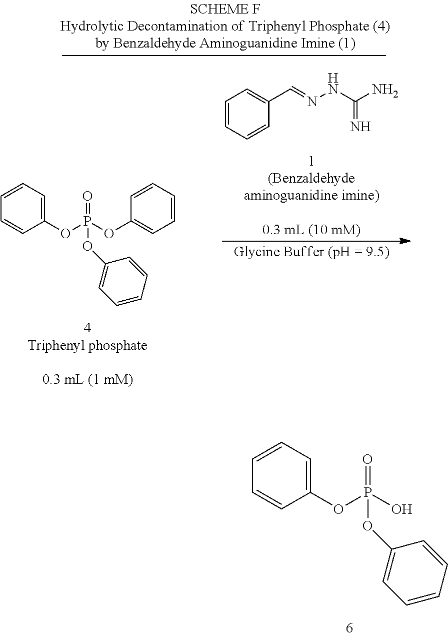 [SMILES: N=C(N)N/N=C/c1ccccc1 ; O=P(Oc1ccccc1)(Oc1ccccc1)Oc1ccccc1 ; O=P(O)(Oc1ccccc1)Oc1ccccc1]

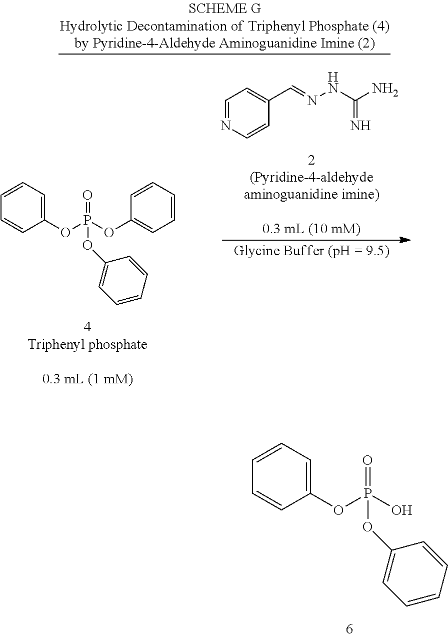 [SMILES: N=C(N)N/N=C/c1ccncc1 ; O=P(Oc1ccccc1)(Oc1ccccc1)Oc1ccccc1 ; O=P(O)(Oc1ccccc1)Oc1ccccc1]

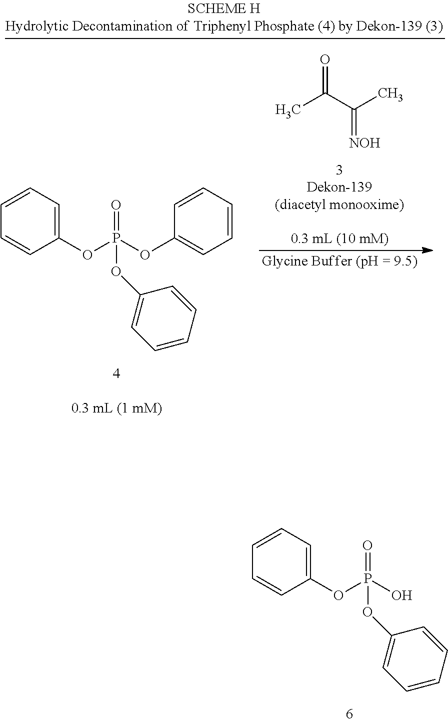

3. Hydrolytic Decontamination of Triphenyl Phosphate at pH˜8.5

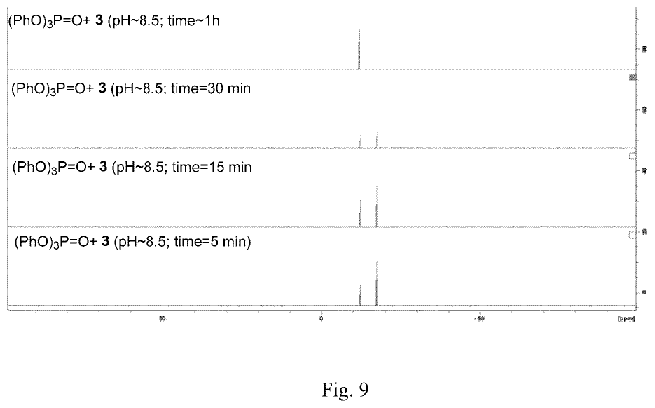

The reaction mixture in this procedure comprised 2.46 mg (0.00765 mmol) of aldehyde imine (1) and 5 mg (0.0153 mmol) of triphenyl phosphate (4) in 2 ml of DMSO (Scheme I). The pH was adjusted to ˜8.5 using aqueous KOH, and the sample was vigorously stirred by a vortex for 2 mins. before the first ³¹P NMR was recorded at 5 mins. The sample was then transferred to a vial and stirred using a magnetic stirred bar. An aliquot was withdrawn and immediately placed in an NMR tube, and the spectra (shown in FIG. 9) were recorded by accumulation of 32 scans.

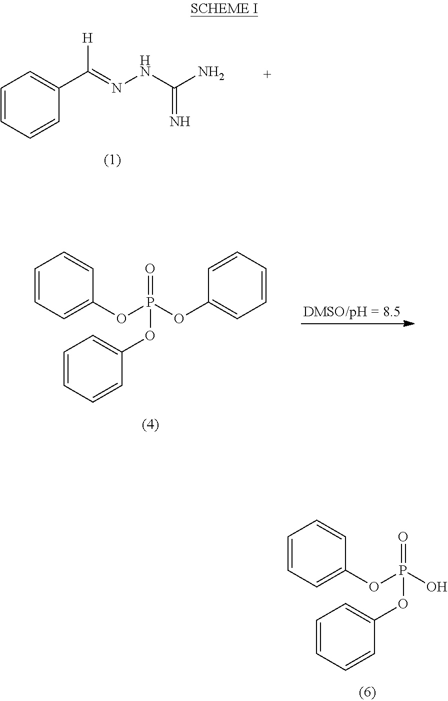 [SMILES: N=C(N)N/N=C/c1ccccc1 ; O=P(Oc1ccccc1)(Oc1ccccc1)Oc1ccccc1 ; O=P(O)(Oc1ccccc1)Oc1ccccc1]

1. A decontamination or neutralization method comprising contacting an organophosphorous compound with an aminoguanidine imine.

2. The method of claim 1, wherein said contacting comprises reacting said organophosphorus compound and said aminoguanidine imine.

3. The method of claim 1, wherein said contacting comprises hydrolyzing said organophosphorus compound.

4. The method of claim 1, wherein said aminoguanidine imine is derived from one or more of an aryl aldehyde, a heteroaryl aldehyde, an aliphatic aldehyde, or combinations thereof.

5. The method of claim 1, wherein said aminoguanidine imine comprises one or more of an aryl aldehyde aminoguanidine imine, an aliphatic aldehyde aminoguanidine imine, a heteroaryl aldehyde aminoguanidine imine, or combinations thereof.

6. The method of claim 1, wherein said aminoguanidine imine is chosen from one or more 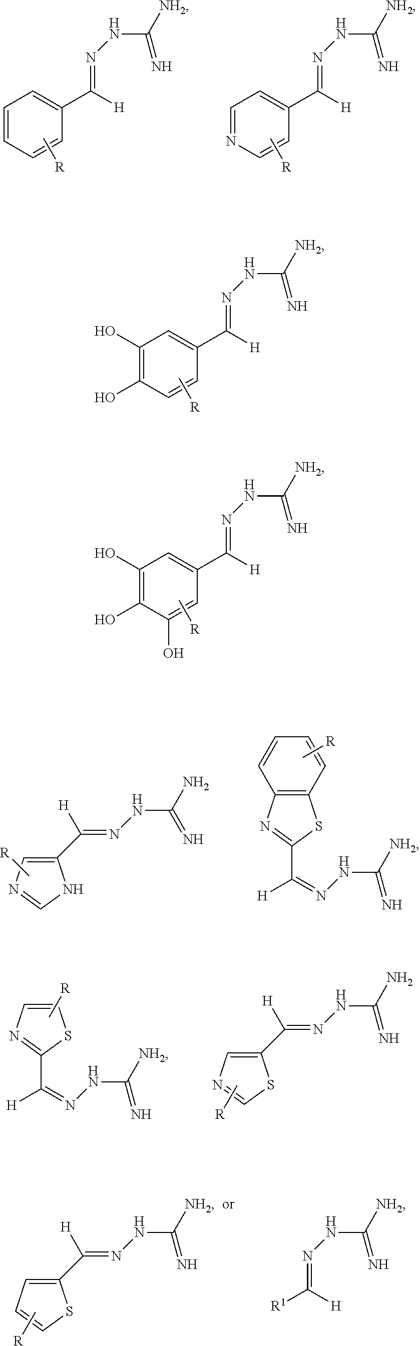
wherein:
R is selected from the group consisting of alkyl groups, aryl groups, —H, and —OR²;
R¹ is selected from from the group consisting of alkyl groups, aryl groups, —H, —OR², and polymers; and
R² is selected from the group consisting of —H and alkyl groups.

7. The method of claim 1, wherein said organophosphorus compound is chosen from one or more of nerve agents, toxins, pesticides, simulants for pesticides, herbicide, or mixtures thereof.

8. The method of claim 1, wherein said organophosphorus compound is present on a surface to be decontaminated.

9. The method of claim 1, wherein said contacting is carried out in a solvent.

10. The method of claim 1, wherein said contacting is carried out in an environment having a pH of less than about 10.3.

11. The method of claim 1, wherein said contacting is carried out in an environment having a pH of about 8 to about 10.

12. The method of claim 1, wherein said contacting is carried out for less than about 5 minutes.

13. The method of claim 2, wherein at least about 90% of said organophosphorus compound is hydrolyzed during said reacting.

14. The method of claim 1, wherein said aminoguanidine imine is present as part of a solution, lotion, kit, gel, or solid.

15. A decontamination or neutralization method comprising contacting an aminoguanidine imine with a surface suspected of contamination or that might become contaminated.

16. The method of claim 15, wherein said aminoguanidine imine is derived from one or more of an aryl aldehyde, a heteroaryl aldehyde, an aliphatic aldehyde, or combinations thereof.

17. The method of claim 15, wherein said aminoguanidine imine comprises one or more of an aryl aldehyde aminoguanidine imine, an aliphatic aldehyde aminoguanidine imine, a heteroaryl aldehyde aminoguanidine imine, or combinations thereof.

18. The method of claim 15, wherein said aminoguanidine imine is chosen from one or more of: 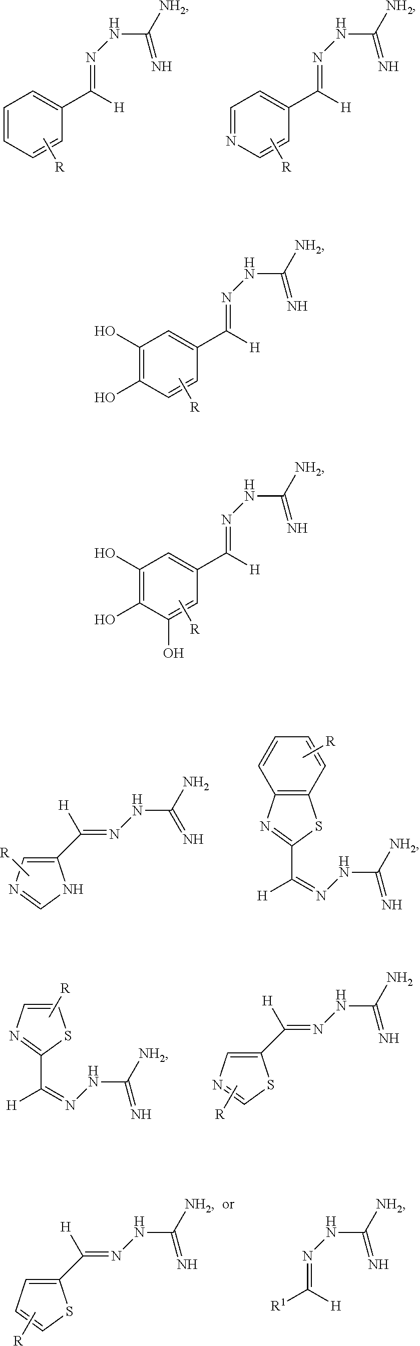
wherein:
R is selected from the group consisting of alkyl groups, aryl groups, —H, and —OR²;
R¹ is selected from from the group consisting of alkyl groups, aryl groups, —H, —OR², and polymers; and
R² is selected from the group consisting of —H and alkyl groups.

19. The method of claim 15, wherein said aminoguanidine imine is present as part of a solution, lotion, kit, gel, or solid.

20. A decontaminant product comprising an aminoguanidine imine.

21. The product of claim 20, wherein said product comprises a solution, lotion, kit, gel, or solid comprising said aminoguanidine imine.

22. The product of claim 20, wherein said aminoguanidine imine is derived from one or more of an aryl aldehyde, a heteroaryl aldehyde, an aliphatic aldehyde, or combinations thereof.

23. The product of claim 20, wherein said aminoguanidine imine comprises one or more of an aryl aldehyde aminoguanidine imine, an aliphatic aldehyde aminoguanidine imine, a heteroaryl aldehyde aminoguanidine imine, or combinations thereof.

24. The product of claim 20, wherein said aminoguanidine imine is chosen from one or more of: 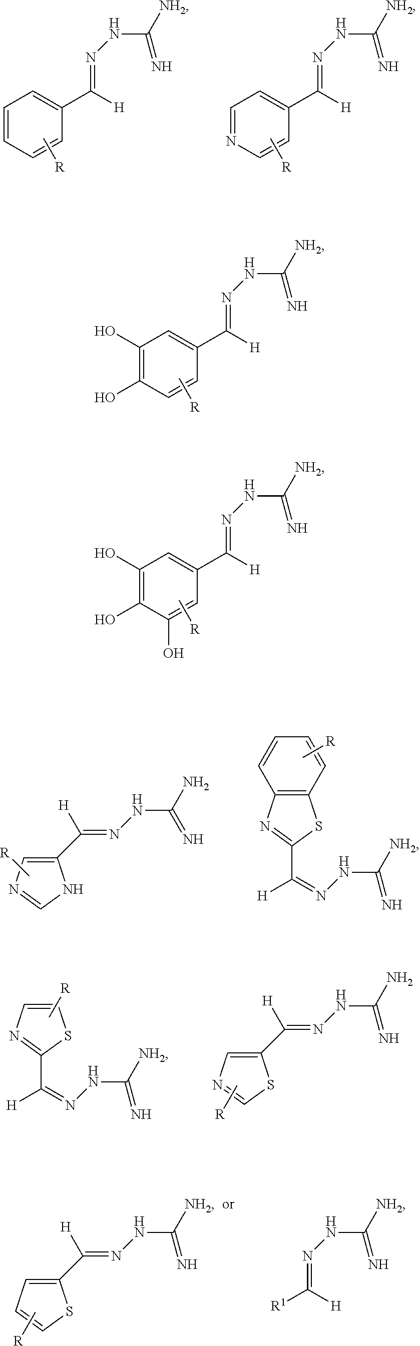
wherein:
R is selected from the group consisting of alkyl groups, aryl groups, —H, and —OR²;
R¹ is selected from from the group consisting of alkyl groups, aryl groups, —H, —OR², and polymers; and
R² is selected from the group consisting of —H and alkyl groups.
